(19) United States
(12) Patent Application Publication
(10) Pub. No.: US 2017/0007261 A1
(43) Pub. Date: Jan. 12, 2017

(54) DEPLOYMENT MECHANISM FOR IMPLANTABLE MEDICAL DEVICES

(71) Applicant: Cook Medical Technologies LLC, Bloomington, IN (US)

(72) Inventors: Rune Tore Paamand, Broenshoej (DK); Raimo Urban Butzbacker, Haslev (DK)

(21) Appl. No.: 15/204,262

(22) Filed: Jul. 7, 2016

Related U.S. Application Data
(60) Provisional application No. 62/190,004, filed on Jul. 8, 2015.

(51) Int. Cl. A61B 17/12

(52) U.S. Cl. CPC A61B 17/12022; A61B 17/12145; A61B 17/12113; A61B 2017/00867

(57) ABSTRACT

A system for controlling deployment of an implantable medical includes an implantable medical device and a gripper mechanism. One or both of the medical device and the gripper mechanism are made from a shape-memory alloy material having a temperature at which the material will transition from a martensite state to an austenite state. The shape-memory alloy material has a transition temperature that is higher than a normal body temperature and a wide hysteresis band, with a temperature at which the material transitions back into the martensite state being below a normal body temperature. The medical device will expand or change shape upon being heated by a current passing therethrough, but will not transition into its remembered shape until such current is applied.

### CROSS-REFERENCE TO RELATED APPLICATIONS

This application claims the benefit of U.S. Provisional Patent Application No. 62/190,004, filed Jul. 8, 2015, the entire contents of which is incorporated by reference herein in its entirety.

### FIELD OF THE INVENTION

The present invention relates generally to implantable medical devices and, more particularly, to a deployment mechanism for expandable implantable medical devices.

### BACKGROUND OF THE INVENTION

Implantable medical devices are well known in the medical field. Such devices include stents, stent-grafts, vena cava filters, embolization coils, and the like. These implantable medical devices are commonly introduced into the target area through an existing body orifice or through an incision made through the patient's skin. The devices are typically delivered in a delivery state using an introducer assembly, where the devices are compressed and constrained by a constraint mechanism, such as a delivery sheath. The delivery sheath having the compressed device therein is introduced into the body, where the medical device is deployed from the delivery sheath.

These implantable medical devices are typically expandable, such that after the device has been deployed from its delivery sheath or the other constraint elements have been removed, the device will be able to transition from the delivery state to a deployed state, where the device is expanded relative to the compressed delivery state.

One way of expanding the device into its deployed state is through the use of self-expanding devices. In this approach, the device can include an outward bias, such that the compression of the device will result in stored potential energy within the device, and the device will automatically expand outward when the constraint elements have been removed. However, such expansion methods can result in undesirable springing or jumping of the medical device out of the introducer assembly. This can result in incorrect placement of the device within the body vessel.

Another type of expansion is balloon expansion. In this approach, an inflatable balloon is included in the introducer assembly. The balloon is typically placed within the device. Once the constraint element has been removed and the device is able to be expanded, the balloon will be inflated, which forces the device to expand outward and into engagement with the body vessel. These types of devices typically do not suffer from the springing or jumping described above in relation to self-expanding devices. However, the inclusion of the balloon can result in a larger introducer assembly, making delivery of the device more difficult. Additionally, inflation of the balloon to expand the device can result in the target body vessel becoming occluded when the balloon is inflated.

Accordingly, improvements can be made in the delivery and deployment of implantable medical devices.

### BRIEF SUMMARY OF THE INVENTION

The present invention provides a medical system for delivering a medical device. The system includes an introducer assembly and a gripper mechanism coupled to the introducer assembly, with the gripper mechanism having both an open state and a closed state. A resistive element is coupled to the introducer assembly and the gripper mechanism, with the resistive element configured for being connected to a current source. A medical device is releasably coupled to the gripper mechanism. The medical device is constructed from a shape memory material having a predetermined deployed shape and being formable into a compressed delivery shape relative to the predetermined deployed shape. The medical device will transition toward the predetermined deployed shape in response to being heated to a final transition temperature and transitioning into an austenite state via the resistive element. The medical device will remain in the austenite state when heating via the resistive element is ceased.

In another approach, a medical system for delivering a medical device is provided, the system comprising: an introducer assembly; a gripper mechanism coupled to the introducer assembly, the gripper mechanism having both an open state and a closed state; a resistive element coupled to the introducer assembly, the resistive element configured for being connected to a current source; wherein the gripper mechanism is at least a portion of the resistive element or is coupled to the resistive element; a medical device releasably coupled to the gripper mechanism; wherein the gripper mechanism includes shape memory material, and the gripper mechanism will transition from a martensite state into an austenite state in response to being heated to a final transition temperature via the resistive element, wherein the final transition temperature is higher than a normal human body temperature.

In another aspect, a medical device is provided including an implantable body configured for being attached to an introducer mechanism including a resistive heating element. The implantable body includes a shape memory material. The body has a predetermined shape in an austenite state and is compressed into a delivery shape in a martensite state. The shape memory material of the body is constructed to transition to the austenite state at a final transition temperature that is above the normal human body temperature and to remain in the austenite state within a hysteresis band. The hysteresis band is defined between the final transition temperature and a lower temperature. The final transition temperature is above the normal human body temperature and the lower temperature is below the normal human body temperature.

In another approach, a method for implanting a medical device within a body vessel is provided, the method comprising the steps of: providing an introducer assembly, a gripper mechanism coupled to the introducer assembly, the gripper mechanism having both an open state and a closed state; a resistive element coupled to the introducer, the resistive element configured for being connected to a current source; and a medical device releasably coupled to the gripper mechanism; wherein the gripper mechanism is at least a portion of the resistive element or is coupled to the resistive element, and the gripper mechanism includes shape memory material, and the gripper mechanism will transition from a martensite state into an austenite state in response to being heated to a final transition temperature via the resistive element, wherein the final transition temperature is higher than a normal human body temperature; delivering the gripper mechanism and the medical device to a body vessel; applying a current to the gripper mechanism and in response thereto, heating the gripper mechanism to the final transition temperature, in response to heating the gripper mechanism to the final transition temperature, transitioning the gripper mechanism to the open state; and in response to transitioning the gripper mechanism to the open state, releasing the medical device from the gripper mechanism within the body vessel.

In another aspect, a method for implanting a medical device within a body vessel is provided. The method includes delivering a medical device to a body vessel, the medical device being in a compressed delivery state, where the medical device comprises a shape memory material configured to transition from martensite to austenite in response to reaching a final transition temperature. The austenite state corresponds to an expanded delivery state and the final transition temperature is above a normal body temperature. The method further includes applying a current through the medical device and in response thereto, heating the device to the final transition temperature. The method further includes, in response to heating the device to the final transition temperature, expanding the device, ceasing application of the current through the medical device, and cooling the device to the ambient body temperature. The device remains in the austenite state and the expanded deployed state in response to being cooled.

### BRIEF DESCRIPTION OF THE DRAWINGS

FIG. 1A is schematic view of a medical device delivery system including a gripper mechanism, a medical device, and a resistive element through which current will pass to cause heating to occur, where one or both of the medical device and gripper mechanism can be made from a shape memory alloy having a transition temperature at which the gripper mechanism and medical device will transition into a preset shape;

FIG. 1B is a schematic view of the medical device delivery system having an alternative arrangement for heating one or both of the medical device and the gripper mechanism;

FIG. 2 is partial schematic elevation view of the gripper mechanism including a pair of arms in a closed position that retain corresponding structure on the medical device;

FIG. 3 is a top schematic view of the gripper mechanism, arms, and corresponding structure of the medical device;

FIG. 4 is a schematic view of the gripper mechanism in an open configuration occurring after the gripper mechanism has reached its transition temperature;

FIG. 5 is a schematic view of an alternative arrangement of the gripper mechanism and corresponding structure of the medical device, with the gripper mechanism shown in the closed position;

FIG. 6 illustrates a filter for use with the system;

FIG. 7 illustrates a stent for use with the system;

FIG. 8 illustrates an embolization coil for use with the system;

FIG. 9 illustrates an alternative embodiment of a connection between the gripper mechanism and the medical device, with the connection in the closed position;

FIG. 10 illustrates the embodiment of FIG. 9 in an open position, with the medical device no longer connected to the gripper mechanism;

FIG. 11 is a schematic view of the system with a stent retained by the gripper mechanism, with the gripper mechanism being closed and the stent in a compressed delivery state;

FIG. 12 is a schematic view of the system with the stent in an expanded state after reaching its transition temperature, and the gripper mechanism being closed and continuing to retain the stent;

FIG. 13 is a schematic view of the system with the stent in an expanded state and the gripper mechanism being opened, and the stent being released from the gripper mechanism;

FIG. 14 is a schematic view of the stent being crimped onto the gripper mechanism for being retained thereon;

FIG. 15 is a schematic view of the stent being in an expanded state after reaching its transition temperature and being free from its attachment to the gripper mechanism;

FIG. 16 is a schematic view of the system with an embolization coil in a delivery state and retained by the gripper mechanism;

FIG. 17 is a schematic view of the system with the embolization coil in a deployed configuration and in its remembered shape after being heated to its transition temperature and being released from the gripper mechanism;

FIG. 18 is a schematic view of an alternative gripper mechanism and the medical device attached thereto;

FIG. 19 is a schematic view of the gripper mechanism of FIG. 18 in an open state with the medical device being released;

FIG. 20 is a schematic view of another gripper mechanism and the medical device attached thereto;

FIG. 21 is a schematic view of the gripper mechanism of FIG. 20 in an open state with the medical device being released;

FIG. 22 is a schematic view of another gripper mechanism and the medical device attached thereto;

FIG. 23 is a schematic view of the gripper mechanism of FIG. 22 in an open state with the medical device being released;

FIG. 24 is an isometric view of an alternative embodiment of the gripper mechanism in a closed state;

FIG. 25 is an isometric view of the gripper mechanism of FIG. 24.

### DETAILED DESCRIPTION OF THE INVENTION

The terms “proximal” and “distal” as used herein are intended to have a reference point relative to the user. Specifically, throughout the specification, the terms “distal” and “distally” shall denote a position, direction, or orientation that is generally away from the user and towards a target site, and the terms “proximal” and “proximally” shall denote a position, direction, or orientation that is generally towards the user and away from a target site. Thus, “proximal” and “distal” directions, portions of a device, or bodily regions, may depend on the point of entry for the procedure (e.g., percutaneously or laparoscopically or endoscopically).

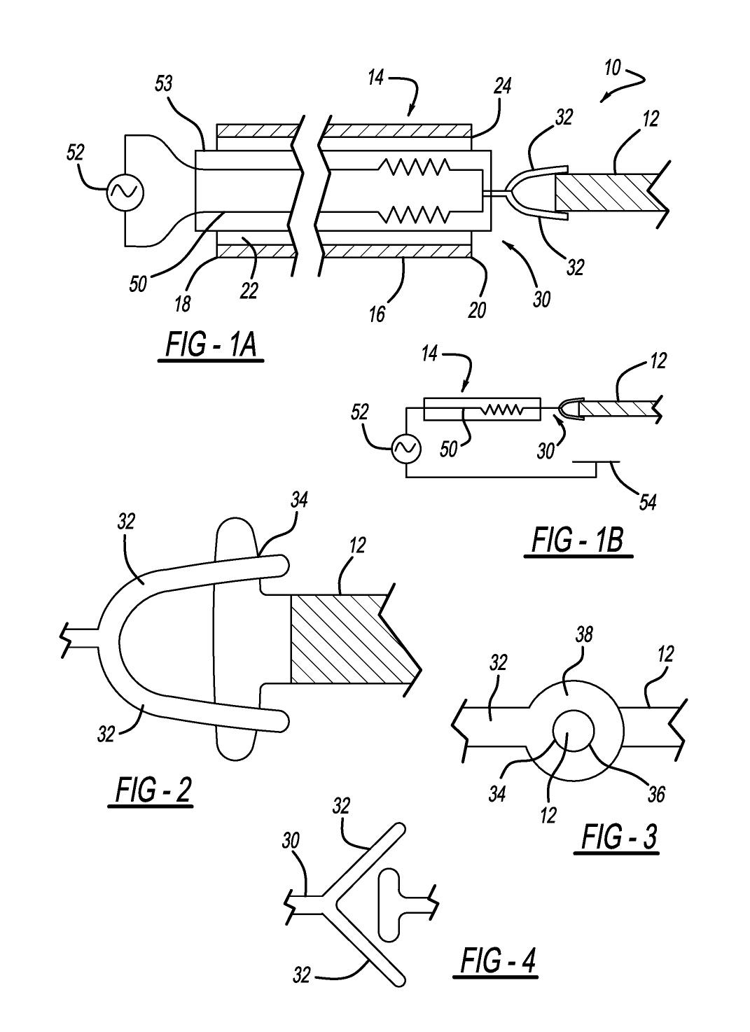

Turning now to the figures, FIGS. 1A and 1B illustrates a schematic of a system 10 including a medical device 12 and an introducer assembly 14. The medical device 12 is releasably mounted to the introducer assembly 14 for being selectively deployed within a body vessel of a patient.

In one approach, the introducer assembly 14 includes a delivery sheath 16 having a proximal end 18 and a distal end 20. The delivery sheath 16 defines a lumen 22 extending from the proximal end 18 to the distal end 20. The distal end 20 includes a distal opening 24 through which the medical device 12 can be exposed from the delivery sheath 16. The proximal end 18 of the delivery sheath 16 can be configured to connect to a hub or handle (not shown) or other mechanism to allow for the sheath to be manipulated by the physician or clinician. In one approach, the introducer assembly 14 can also include a guidewire (not shown) for assisting in the delivery of the sheath 16 to the target site, where the delivery sheath and medical device 12 can be introduced over the guidewire.

The introducer assembly 14 can further include a gripper mechanism 30 sized and configured to grip or otherwise hold the medical device 12 in place prior to being deployed. In one approach, the gripper mechanism can 30 can include a pair of arms 32 that are sized and configured to mate with corresponding structure of the medical device 12 in order to hold the medical device 12. The arms 32 are moveable from a closed position, in which the arms 32 retain the medical device 12, to an open position, in which the arms 32 release the device 12.

With reference to FIGS. 2 and 3, in one approach, the arms 32 can each define a recess 34, into which corresponding structure of the medical device 12 can be inserted. Accordingly, when the arms 32 are closed, the medical device 12 cannot be removed from the gripper mechanism 30. In one form, the recess 34 can be in the form of a hole 36 defined by a ring 38.

In another approach, as shown in FIG. 5, the arms 32 can each include a post 40, such that the posts 40 of the arms 32 extend toward each other to create a pincer-style retention of the medical device 12. The posts 40 may contact each other when the arms 32 are in the closed position, or the posts 40 can remain spaced apart or not in contact with each other in the closed position. In either case, when the arms 32 open, the posts 40 will move away from each other to release the medical device 12 from its retention by the gripper mechanism 30.

Referring once again to FIGS. 1A and 1B, the gripper mechanism 30 can be connected to or include a resistive element 50. The resistive element 50 can be part of the introducer assembly 14. The resistive element 50 or introducer assembly 14 can be coupled to a current source 52. Accordingly, a current can be generated by the current source 52 and applied to the resistive element 50, which will in turn be applied to the gripper mechanism. Application of the current to the resistive element 50 and the gripper mechanism 30 will cause the resistive element 50 and gripper mechanism 30 to increase in temperature, which can assist in the deployment of the medical device, as further discussed below. The resistive element 50 and gripper mechanism 30 can include an insulating cover 53 around at least a portion of the gripper mechanism 30 and resistive element 50.

FIG. 1A illustrates an arrangement where current will flow through the resistive element 50 and cause the gripper mechanism 30 and the arms 32 to become heated. More particularly, the arms 32 will become heated via conductive heating and heat transfer from the resistive element.

FIG. 1B illustrates an alternative arrangement where current will flow through the resistive element 50, gripper mechanism 30, medical device 12, and through the patient's body via a grounding pad 54 that completes the circuit.

In FIGS. 1A and 1B, the resistive element 50 is illustrated schematically as being in the form of a wire or other conductive element with a pair of resistors as part of the circuit. It will be appreciated that the resistive element 50 can be free from the inclusion of resistors and the conductive wire itself can have a resistance sufficient to result in the desired increase in temperature.

The gripper mechanism 30 can be made from a shape memory material or shape memory alloy (SMA) material, for example Nitinol. Shape memory material can be set to a predetermined shape and re-formed into other shapes. Upon reaching a transition temperature, the material will return to its predetermined remembered shape.

When the shape memory material is in a deformed shape that is not the predetermined shape, the material is in a martensite state. As the material is heated, it stays in the martensite state until it reaches its transition temperature, where it will transition into an austenite state, such that the material will return to its predetermined remembered shape. When the material cools, it will return to the martensite state, where it is not biased toward its predetermined remembered shape and can be reformed or molded to other shapes in response to external forces applied thereto.

When the gripper mechanism 30 is made from a shape memory material, it can therefore be controlled by controlling the temperature of the gripper mechanism 30. The gripper mechanism 30 can be loaded in a closed position in the martensite state. When the introducer assembly 14 is placed into the body with the medical device 12 attached, the gripper mechanism 30 will continue to hold the medical device 12 until the gripper mechanism is heated to its transition temperature.

In one approach, the transition temperature of the gripper mechanism is selected to be a temperature above a temperature that would typically correspond to a temperature that the patient's body could not naturally tolerate. Thus, merely placing the gripper mechanism 30 into the patient's bloodstream would not cause the gripper mechanism 30 to transition to its remembered state. For example, in humans, hyperpyrexia occurs at about 40 degrees C., and body temperatures at this level are considered life threatening. Accordingly, a transition temperature of 40 degrees C. is unlikely to be reached simply by being placed in the patient's body, and would typically require an additional source of heat for the gripper mechanism 30 to reach a transition temperature that is above normal body temperatures.

As used herein, normal body temperatures refer to temperatures in the body that occur naturally and are typically non-life threatening. For example, 98.6 degrees F. is a common normal body temperature in a human, and a fever of 100 degrees F. would also be a normal body temperature. A fever of 106 degrees F., on the other hand, is not a normal body temperature.

Thus, in one approach, the arms 32 of the gripper mechanism 30 could have a predefined shape corresponding to the open state of the gripper mechanism 30, with a transition temperature of about 40 degrees C. Of course, other transition temperatures above normal body temperatures could also be used.

As stated above, the gripper mechanism 30 is connected to a resistive element 50 that is connected to a current source 52. Thus, by supplying a current through the resistive element 50 and the gripper mechanism 30, the gripper mechanism 30 will be heated. The amount of current to be supplied can depend on the material properties of the resistive element 50, the resistance value of the resistive element 50, the desired temperature, etc. The current to be supplied is selected such that the transition temperature of the gripper mechanism 30 and the arms 32 is reached.

Upon a sufficient supply of current and the resulting heating of the gripper mechanism 30 beyond the transition temperature, the arms 32 will transition to the austenite state (the remembered shape) and into the open position, releasing the medical device 12.

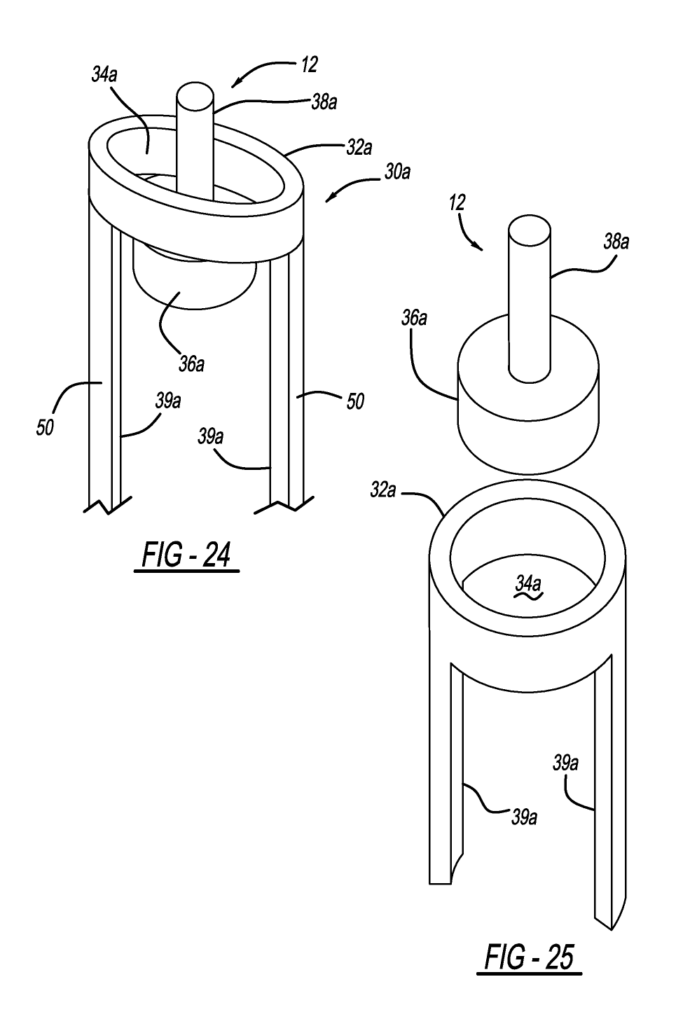

In one embodiment, and with reference to FIGS. 24 and 25, an alternative gripper mechanism 30a can be used to retain the medical device 12. The gripper mechanism 30a, similar to the gripper mechanism 30, can have both an open position and a closed position. However, unlike the gripper mechanism 30, the gripper mechanism 30a does not include arms. Rather, the gripper mechanism includes a ring 32a that can change its shape to selectively retain a corresponding structure of the medical device 12 or release the corresponding structure of the medical device 12.

The ring 32a can define a closed loop that defines an opening 34a or through-hole, and has an open position where the shape of the ring 32a is generally circular, although an oval or ellipse shape could also be used for the open position. In the closed position, the shape of the ring 32a is compressed to reduce the width of the opening relative to the open position. In the case of the open position being a circle, the closed position can be an oval or ellipse. In the case of the open position being an oval or an ellipse, the closed position can be a narrower oval or ellipse, such that the distance across the opening 34a is reduced along one axis and the distance across the opening 34a is extended along a transverse axis. In another approach, the ring 32a could be compressed along multiple axes to reduce the distance across the opening 34a in multiple locations of the ring 32a.

In this embodiment, the medical device 12 can include structure corresponding to the ring 32a, where the structure is in the form of a head portion 36a having a neck portion 38a. The head portion 36a can be enlarged relative to the neck portion 38a. The head portion 36a is sized such that it can pass through the opening 34a of the ring 32a when the ring 32a is in the open position, but will be retained and prevented from passing through the ring 32a when the ring 32a is in the closed position.

The gripper mechanism 30a can also include a pair of posts 39a extending from the ring 32a. The posts 39a, in one approach, are disposed on diametrically opposed points of the ring 32a. However, the posts 39a could be connected to other locations on the ring 32a that are not diametrically opposed. In the closed position of the ring 32a, the posts 39a are further apart than they are in the open position of the ring 32a. In the closed position, the head portion 36a of the medical device 12 can be disposed laterally between the posts 39a. In the open position, the posts 39a move closer to the head portion 36a, and the head portion 36a will still fit between the posts 39a prior to the head portion moving out of the ring 32a.

In one approach, the gripper mechanism 30a and the posts 39a thereof can be coupled to the resistive element 50, such that heat from the resistive element 50 will heat the posts 39a via conductive heating, causing the temperature of the gripper mechanism 30a to increase, where the ring 32a will open.

In another approach, the gripper mechanism and the posts 39a can be part of the resistive element 50 such that the current applied to the resistive element will flow through gripper mechanism 30a, with the gripper mechanism 30a providing at least some of the resistance in the circuit, thereby causing the gripper mechanism 30a to become heated and the ring 32a to open. For example, the resistive element 50 can include a pair of leads in the form of a wire or the like, with the leads being made of a material with high conductivity and low resistivity, such as copper. In this approach, the current can flow through the leads to and from the gripper mechanism 30a, with the gripper mechanism 30a itself having a relatively high resistance, thereby becoming heated.

In another approach, the posts 39a can define the resistive element 30, where the posts 39a are the leads of the resistive element, and the material of the leads/posts 39a has a sufficiently high resistance to be heated to a target temperature in response to a current flowing through the posts 39a and the gripper mechanism 30a.

The above described examples of the gripper mechanism 30a being the resistive element of the circuit can also be applied to the other gripper elements 30 described herein, with low resistivity leads and a relatively high resistivity gripper element 30. Similarly, in the case where the gripper mechanism defines the leads of the circuit, this can also be applied to the other gripper mechanisms 30, where the leads and the gripper mechanism are made of a resistive material.

In the case where the gripper mechanism 30/30a is the resistive element and the leads have a low resistance, the transition temperature of the gripper mechanism 30/30a (where it will transition from a closed state to an open state due to the shape memory characteristics of the material) can be substantially higher than the examples described herein where the resistive element 50 is heated within the delivery device, or when the medical device 12 is part of the circuit or otherwise heated. The higher temperature can be used to transition the gripper mechanism 30/30a in this embodiment without overheating tissue adjacent the medical device 12 or the delivery device. In this approach, for example, the transition temperature of the gripper mechanism 30/30a could be up to approximately 70 degrees C. Further, the selection of a particular transition temperature in this embodiment can be less strict when targeting a high transition temperature, because even if the transition temperature is below the intended transition temperature, the actual transition temperature will still be above a normal body temperature, and the gripper mechanism 30/30a will not release in response to simply being placed in the body.

For the sake of further discussion, the various gripper mechanisms 30, 30a will be referred to generally with reference numeral 30.

Shape memory alloys also include a property known as hysteresis in addition to the transition temperature. When the material has been heated to its transition temperature and has transitioned to the austenite state, the material will remain in the austenite state even when the temperature drops below the final transition temperature. The material will transition back to martensite at a temperature lower than the final transition temperature. The gap between the final transition temperature and the temperature at which the material converts back to martensite (and capable of being reformed into other shapes) is known as a hysteresis band.

Thus, even after heating the material has ceased, the gripper mechanism 30 will not immediately return to martensite. It will reach the martensite state only after it has cooled to the lower boundary of the hysteresis band. Thus, after heating has ceased, if an external force is applied to the gripper mechanism 30 that may cause the gripper mechanism to be forced toward a closed state, the gripper mechanism 30 will resist that external force, and return to the remembered open shape as long as the gripper mechanism 30 remains at a temperature within the hysteresis band after initially reaching the transition temperature.

Furthermore, shape memory metals may exhibit superelasticity in the austenite phase. This is a much desired property in that it provides kink-resistance to wires and flexibility of devices. A wide hysteresis band will ensure that a device will remain superelastic after deployment. Superelasticity is described in further detail below.

In one approach, the gripper mechanism 30 is made from a shape memory material that has a relatively large hysteresis band, with a lower boundary that is below a normal body temperature. Thus, even as the material within the body cools, it will generally plateau around the temperature of the patient's body. With the lower boundary temperature being below normal body temperature, the material will remain in the austenite state and the predetermined shape and be in a superelastic state, where external forces may be applied that will not cause the shape of the material to change permanently. Thus, once the gripper mechanism 30 has been placed within the body and heated, it will move to the open state, and will remain in the open position even after cooling to the temperature of the body.

Accordingly, the use of shape memory material for the gripper mechanism 30 results in a reliable method of releasing the medical device 12 from the introducer assembly 12, thereby improving the reliability of the placement of the device, as the device can be positioned appropriately prior to applying the heating element. This particular embodiment of the gripper mechanism 30 also allows the gripper mechanism 30 to be generally free from complicated mechanisms or pivoting jaws requiring mechanical actuation.

It will be appreciated, however, that a wide hysteresis band for the gripper mechanism can be replaced with a more narrow or traditional hysteresis band when using the gripper mechanism 30 to release the medical device 12 and still result in effective release of the medical device 12. Once the medical device 12 is released, the gripper mechanism 30 being deformed to a non-open position does not affect the deployment of the medical device.

The above description regarding the use of shape memory material for the gripper mechanism 30 can also apply to the use of shape memory material for the medical device 12, which can also be beneficial for medical devices 12 that have a deployed shape that is different from its shape during delivery of the device 12 to the target location.

Heat is capable of being applied to one or both of the medical device 12 and the gripper mechanism 30 without the need for a separate heating circuit that runs in parallel with the medical device 12 and gripper mechanism 30. Rather, current passes through the gripper mechanism 30 and/or medical device 12 itself. In one approach, for example, the gripper mechanism 30 and/or medical device 12 are configured as an “RF-device” where 500 kHz current, in one example, is passed through the device 12 and/or mechanism 30 itself and with a return path through a grounding pad on the patient or a proximal electrode region on the delivery system. Of course, the above disclosed current is merely exemplary, and other frequencies can also be used to generate heat, as known in the art. In another approach, a grounding pad could be omitted and using a higher frequency current, using the self-capacitance of the patient as the “return,” similar to a hyfrecator. In this approach, the surface of the device 12 or gripper mechanism 30 will heat up, as the interface with the patient's tissue has high current density and high resistance.

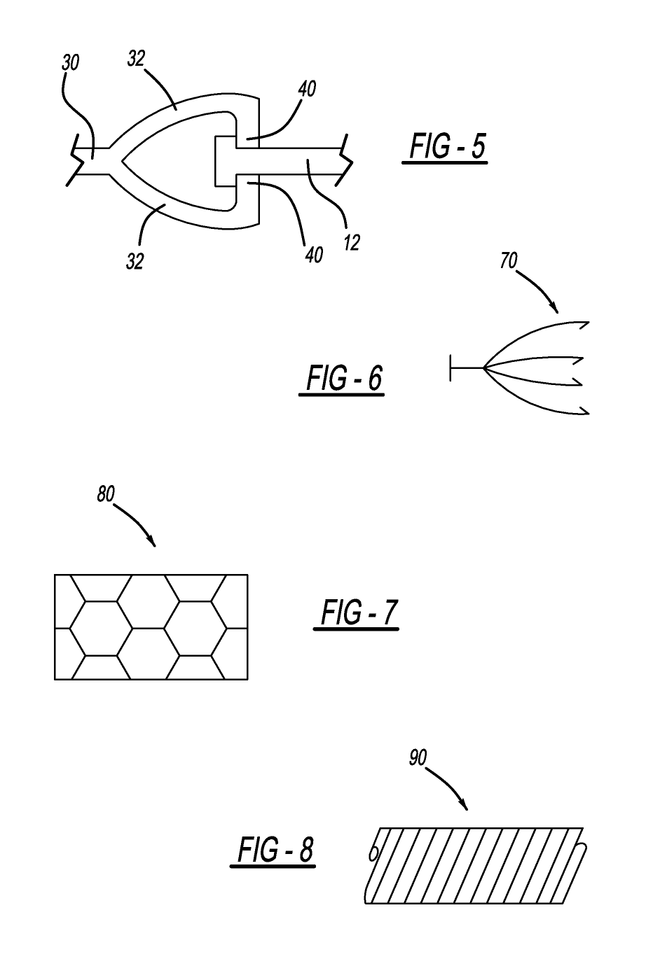

The medical device 12 can be one of many known implantable medical devices that are typically delivered to a body lumen via a typical introducer assembly. For example, as shown in FIGS. 6-8, the medical device 12 can be a filter 70, a stent 80, an embolization coil 90, or the like. The medical device 12 can also be in the form of a combination of known medical devices, such as a combination filter and stent, or a stent-graft. It will be appreciated that various other types of implantable medical devices could also be used. The medical devices 12 can be of the expandable type, either self-expanding or manually expandable, such as via balloon inflation, (for example, stents and filters) or they can be non-expandable (for example, embolization coils) where the device implants into the body vessel in another manner. For example, an embolization coil may be configured to fill a body vessel or body cavity in a random tortuous manner due to the inherent springiness of the coil, where the coil will fold over itself without necessarily expanding, or the coil could have shape memory characteristics, further described below, where the coil has a remembered shape.

The above description has referred to the gripper mechanism 30 releasing the medical device 12 after the gripper mechanism 30 has been heated to its transition temperature. In the approach described above, the arms 32 of the gripper mechanism 30 move outward to release the device 12, or the ring 32a changes its shape to the open position to release the device.

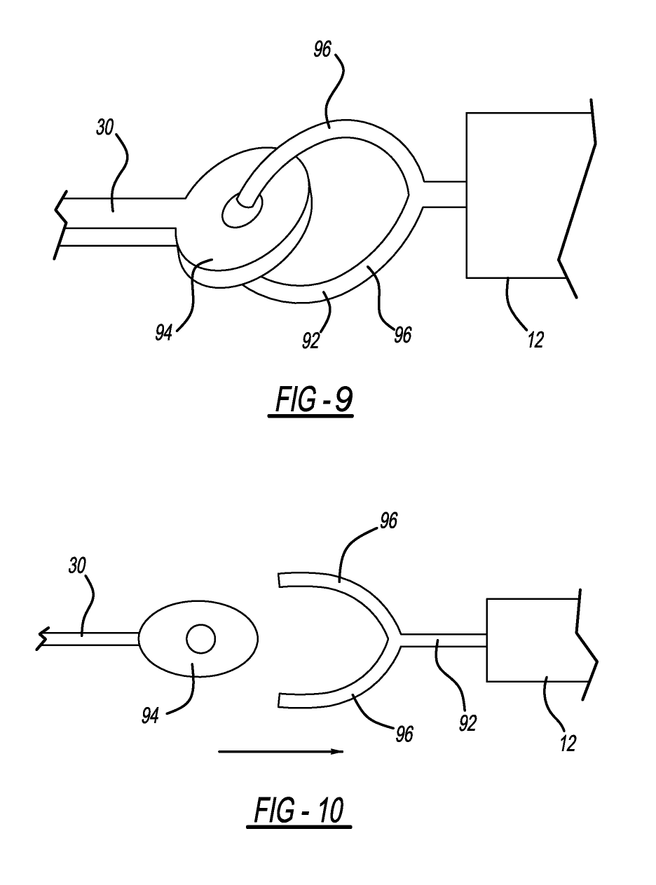

In another approach, as shown in FIG. 9, the medical device 12 itself may include a proximal release mechanism 92. The release mechanism 92 is coupled or attached, either directly or indirectly, to a distal end of the gripper mechanism 30. Instead of the gripper mechanism 30 including moveable arms that move in response to being heated to a transition temperature, the gripper mechanism includes a fixed distal coupling 94, and the release mechanism 92 of the medical device 12 includes arms 96. The above described embodiments of the arms 32 and corresponding connection of the medical device 12 can be applied to this alternative arrangement, with the various arm types being applied to the release mechanism 92 and the corresponding structure being applied to the fixed distal coupling 94.

Similar to the above description of the gripper mechanism 30 providing the resistance in the circuit, the fixed distal coupling 94 can provide the resistance, where the current flows through the fixed distal coupling 94. In this approach, low resistance leads can be used, reducing the heat generated in the delivery device. In yet another approach, the distal coupling 94 can be a part of the resistive element 50, where the leads provide the resistance.

The gripper mechanism 30 can remain connected to or including the resistive element 50, such that current will still pass through the gripper mechanism 30. As described above, there are various ways of causing the distal coupling 94 to become heated. Upon application of current to the gripper mechanism 30, the release mechanism 92 of the medical device 12, being coupled to the distal coupling 94 of the gripper mechanism 30, will likewise be heated by the current and will approach its transition temperature. When the release mechanism 92 reaches its transition temperature, the arms 96 of the release mechanism 92 will move to their open position, releasing the arms 96 and the medical device 12 from the distal coupling 94, as shown in FIG. 10.

The above described embodiments for releasing the medical device 12 from the gripper mechanism 30 can be applied to any known medical device that is self-expanding, manually expanded, or non-expanding. Thus, the above described manner of releasing the medical device 12 can be applied to any device where selectively releasing the device is desired. This manner of releasing the device 12 can be used as an alternative to traditional release mechanisms, such as mechanically actuated release mechanism or by using an intermediate sheath to expose an expandable device. Thus, the above described manner of releasing the device can result in a simpler release mechanism with fewer moving mechanical parts, or it can include a reduced outer profile by eliminating the intermediate sheath, if desired.

In each of the above described release arrangements, the medical device 12 will be released upon the arms 32 or 96 or the ring 32a being heated to their transition temperature. The use of a transition temperature above normal body temperature and a wide hysteresis band, where transition back to the martensite state will only occur at a lower than normal body temperature, results in selective release of the medical device 12. This also allows the arms 32 or 96 to expand and resisting closing immediately thereafter, because they will only be allowed to be permanently closed upon falling to a temperature that is below normal body temperature and transitioning back to a martensite state. Until that point the arms 32 or 96 will remain biased toward the remembered open state. As described above, the gripper mechanism 30 can also be used with a smaller or narrower hysteresis band and still release the medical device 12.

Upon release of the medical device, the medical device 12 can be manually expanded, such as via an inflatable member, self-expanded, such as via stored potential energy where the device 12 will spring outward, or it can release and be allowed to wind and fold to fill a body cavity in the case of an embolization coil. In the case where the medical device 12 is made of a shape memory material, the device 12 can expand or change its shape upon being heated to its transition temperature.

Further reference will be made to the gripper mechanism 30 being the element that opens to release the medical device 12, but it will be appreciated that such references can also apply to the medical device 12 having the release mechanism 92 with arms 96.

In addition to using SMA material as part of the gripper mechanism 30, the SMA material could also be used to form the medical device 12, as mentioned above. For example, the filter 70, stent 80, or coil 90 could be made from the SMA material having the high transition temperature and wide hysteresis. The medical device 12 can be made of this SMA material in addition to the gripper mechanism 30 or as an alternative to the gripper mechanism 30.

In the case where both the medical device 12 and the gripper mechanism 30 include the SMA material, the medical device 12 can have a remembered shape into which the device 12 will transition when it has reached its transition temperature. For example, in the case of an expanding filter 70 or stent 80, the remembered shape is the expanded shape. In this approach, the medical device 12 is loaded into the introducer assembly 14 in a compressed delivery state (for filters and stents) or an elongate delivery state (for coils) in the generally stiff and formed martensite state, and will transition to a more flexible and expanded or coiled state upon transitioning to the remembered austenite state.

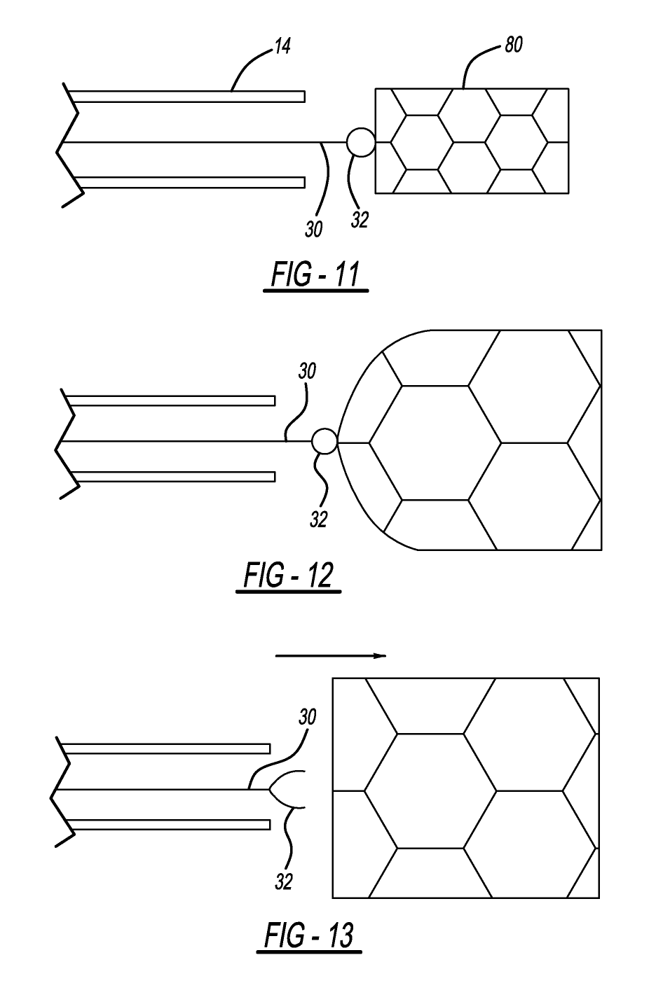

An example of the compressed delivery state is illustrated in FIG. 11, which illustrates the stent 80 being in a compressed shape and yet to be heated to its transition temperature. For purposes of illustration, the stent 80 will be shown in various compressed and expanded shapes, but it will be appreciated that the same illustrated concepts can be applied to the filter 70 or other similar expandable medical device.

In this approach, the medical device 12 is heated via a current running through the device 12 itself, with the current flowing through the device as described above, using a grounding pad or the patient's body as the “return.” Accordingly, a connection to the power source 52 such that current will flow through the device 12 is maintained until the desired transition temperature is reached. Thus, the medical device 12 and the gripper mechanism 30 can include different transition temperatures. The medical device 12 will have a first transition temperature that is above normal body temperature. The gripper mechanism 30 will have a second transition temperature that is higher than the first transition temperature of the medical device. In one approach, the first transition temperature of the medical device 12 is around 40 degrees C., with the second transition temperature of the gripper mechanism 30 being around 45 degrees C.

Thus, by running current through the gripper mechanism 30 and the attached medical device 12, both the gripper mechanism 30 and the medical device 12 can be heated at approximately the same rate. When the medical device 12 and gripper mechanism 30 become heated to the first transition temperature, the medical device 12 will expand into its remembered shape, as shown in FIG. 12, but will remain connected to the gripper mechanism 30. Because the first transition temperature is lower than the second transition temperature of the gripper mechanism 30, the gripper mechanism 30 will continue to retain the expanded medical device 12.

The medical device 12 and gripper mechanism 30 will remain connected to each other and will continue to be heated and increase in temperature. Upon reaching the second transition temperature, the gripper mechanism 30 will release the expanded medical device 12, as shown in FIG. 13.

The above description has referred to using SMA material for the gripper mechanism 30 and medical device 12 that have different transition temperatures. This results in a staggered expansion of the device 12 and release of the device 12. However, the same staggered approach can be obtained by using the same SMA material but altering the resistance for each element. In this approach, the medical device 12 and gripper mechanism 30 can have the same transition temperature, but the medical device 12 will reach the transition temperature first such that it will expand prior to being released.

Similarly, the medical device 12 and gripper mechanism 30 can have different transition temperatures in combination with different resistances to control the ordered expansion and release of the medical device 12. It will be appreciated that various modifications along these lines can be made to suit the needs of the user.

In an alternative approach, the gripper mechanism 30 can be free from moveable arms or otherwise arranged to be generally fixed in its shape in response to a temperature increase. In this approach, the gripper mechanism 30 can be made from a SMA material but having a remembered shape that remains the same, or the gripper mechanism 30 can be made from a non-SMA material. In a preferred approach, the gripper mechanism 30 remains conductive regardless of material such that current will pass through it.

In this approach, with the gripper mechanism 30 having a fixed shape, the gripper mechanism 30 can be considered as a delivery mechanism or gripped member, where the medical device 12 can be crimped onto the gripper mechanism 30 and released simply upon expansion without any substantial change in the shape of the gripper mechanism 30.

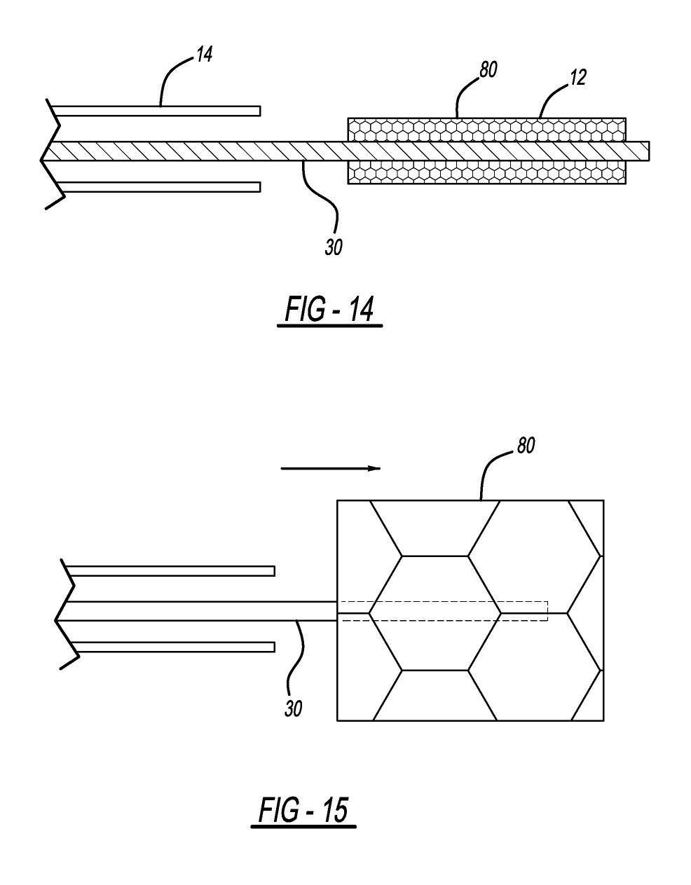

For example, in this approach, as shown in FIG. 14, the medical device 12 can be, in one form, the stent 80. In this approach, the stent 80 is made from SMA material having a high transition temperature and wide hysteresis, as previously described. The stent 80 is crimped onto the distal end of the gripper mechanism 30 such that the gripper mechanism 30 will carry the stent 80 thereon as the gripper mechanism 30 is advanced into the body vessel. The stent 80 will remain in a compressed state even after being exposed from the delivery sheath 16.

Similar to the above, the gripper mechanism can be heated by the application of current. The heat of the gripper mechanism 30 can transfer through the gripper mechanism 30 and into the stent 80 via conductive heating. The stent 80 will increase in temperature and approach its transition temperature. Upon reaching the transition temperature, the stent 80 will expand into its remembered shape, as shown in FIG. 15, and toward engagement with the body vessel wall. The stent 80 will therefore be free from its crimped connection to the gripper mechanism 30, and the gripper mechanism 30 can be retracted.

In a manner similar to an above described embodiment, the crimped connection can also be used with the gripper mechanism having a moveable gripping element or clamp that is configured to open at a different time during the heating process. Unlike the above described stepped release approach, in this approach, the gripper mechanism 30 is configured to release its clamp first, with the stent 80 expanding after the clamp has been released. Thus, the crimp of the stent 80 on the gripper mechanism still controls when the stent 80 will expand and release, but the clamp can provide an added connection element to increase the reliability of the connection between the stent 80 and the gripper mechanism 30 during delivery.

The above described use of medical devices 12 made of SMA with high transition temperatures and wide hysteresis can be beneficially used for medical device 12 that are intended to expand, such as filters, stents, and the like.

However, the use of SMA with high transition temperature and wide hysteresis can also be beneficial for other medical devices 12 where their implanted shape differs from its shape during delivery, such as embolization coils 90.

Traditional embolization coils 90 are typically designed to fold over themselves in a random manner in order to fill a body cavity and promote embolization therein. This tends to occur via a built up spring force in the coil, such that this folding over will occur once released. However, this can make placement of the embolization coil difficult because of its tendency to convert into its folded shape, and it can further be unpredictable.

Thus, the use of SMA material with high transition temperature and wide hysteresis can therefore be beneficial for use with the embolization coils 90, where the coil 90 can have a remembered shape and the transition to the remembered shape can be controlled by selectively applying heat.

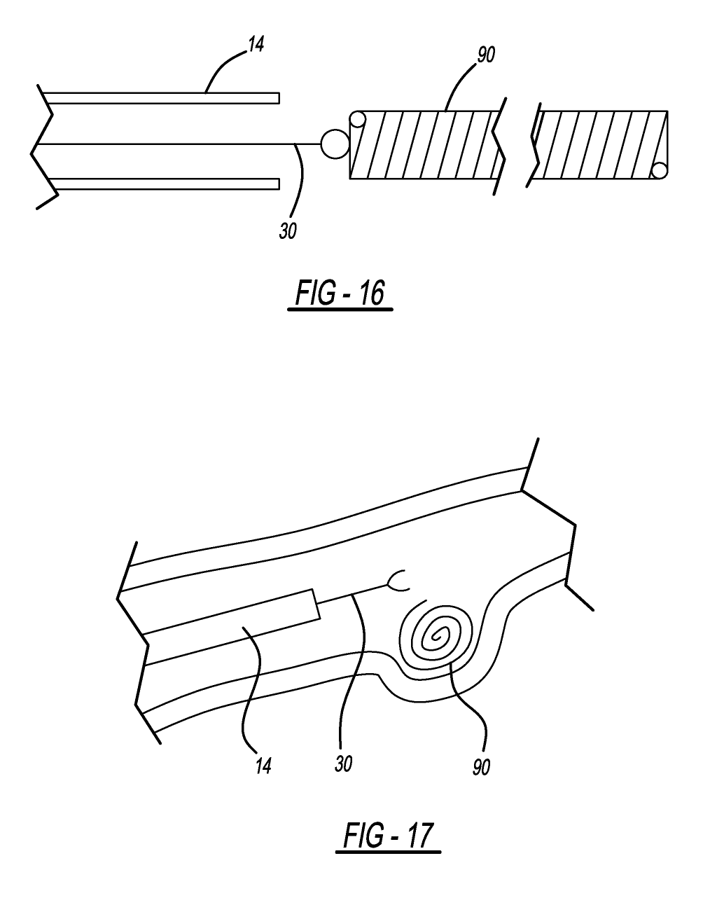

With reference to FIG. 16, in this approach, the embolization coil 90 can be attached to the gripper member 30 in a manner described previously above. Prior to being heated, the coil 90 can remain in its generally straight delivery state, as shown in FIG. 16. Upon being heated, the coil 90 will approach its transition temperature and be released from the gripper mechanism 30. Upon reaching the transition temperature, the coil 90 will move into its remembered shape, as shown in FIG. 17, which can correspond to any predetermined shape. In one approach, the predetermined shape is a tight ball. However, other shapes could also be used, such as an egg-shape, oval-shape, elliptical-shape, bulbous-shape, or any other desired shape. The predetermined shape can be configured and set to correspond to the shape of the particular body cavity where embolization is desired.

Additionally, a typical embolization coil is formed from a single wire that is itself coiled into a larger elongate wire shape, giving the wire a spring-like form. In the disclosed approach, the embolization coil 90 could be made similarly, or it could be made from a single wire. The memorized shape of the single wire can result in the desired shape without requiring a spring-like structure to cause it to fold over itself or otherwise spring into a different deployed shape.

In each of the above described embodiments where the medical device 12 is made from SMA material with high transition temperature and wide hysteresis, upon being heated and transitioning, the medical device 12 will continue to remain in the remembered shape or be biased toward the remembered shape even after the application of heat has ceased, due to the wide hysteresis band. Accordingly, stents and filters will maintain their outward force against the vessel wall after the application of heat has ceased and the device 12 has cooled to body temperature. Similarly, embolization coils 90 will remain in their remembered shape and will resist being reshaped in response to application of exterior force after cooling to normal body temperature.

As described above, in the austenite state, shape memory materials exhibit superelasticity. When the medical device 12 has a superelastic condition after reaching the final transition temperature, the medical device 12 can therefore bend and flex in response to external forces exerted thereon and will return to the predetermined shape of the austenite state when the external forces are removed. The medical device can remain in the superelastic condition after heating of the medical device 12 has ceased and the temperature of the device 12 has fallen below the final transition temperature due to the hysteresis band. The medical device 12 can remain in the superelastic condition at temperatures above a predetermined lower temperature of the hysteresis band. Further the predetermined lower temperature of the hysteresis band can be below a normal body temperature, thereby allowing the medical device to retain the superelastic capabilities after deployment.

The superelastic capabilities would also apply to the gripper mechanism 30 after being heated to the transition temperature and while the temperature of the gripper mechanism remains within the hysteresis band (whether the band is large is small). However, with the gripper mechanism 30 generally does not remain in the body as long as the medical device 12, so a loss of superelasticity if the gripper mechanism 30 cools will generally not affect performance of the system, and a smaller hysteresis band for the gripper mechanism 30 may be desirable.

The above described manner of deploying these devices does not preclude removal of the devices using traditional methods.

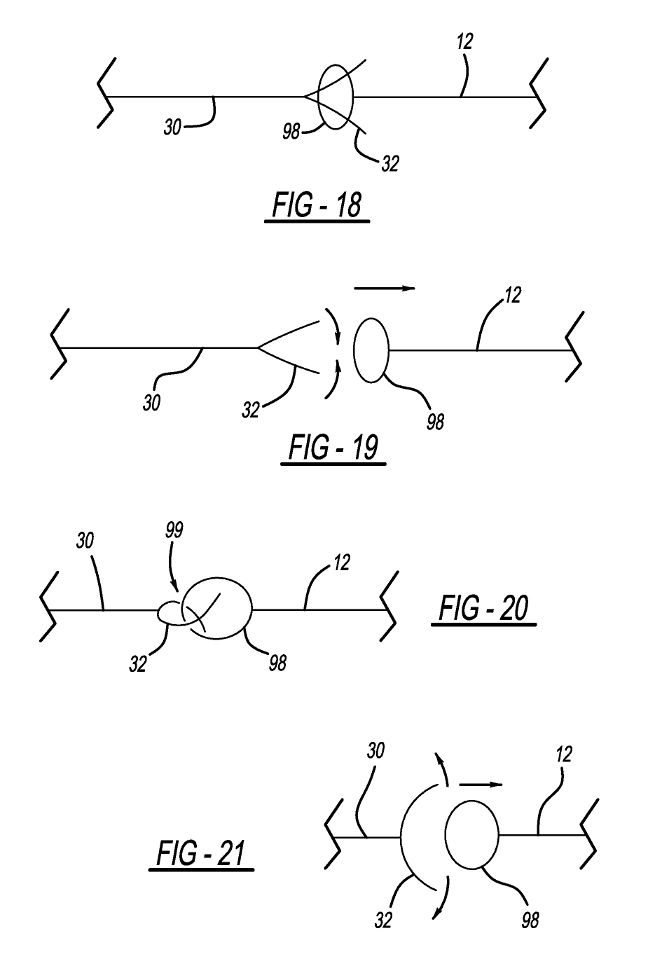

As described above, the gripper mechanism 30 can have different shapes and configurations to release the medical device therefrom. In another approach, as shown schematically in FIGS. 18 and 19, the arms 32 can begin in an open configuration and extending through a loop 98 in the corresponding structure attached to the medical device 12. Upon reaching transition temperature, instead of opening, the arms 32 will move toward each other, allowing the loop and medical device 12 to slide off the arms 32, as shown in FIG. 19.

In another approach, shown in FIGS. 20 and 21, the arms 32 can be bent toward each other and extend across each other to define a closed loop 99. Upon reaching a transition temperature, the arms 32 will release the loop 99, allowing the medical device 12 to become free.

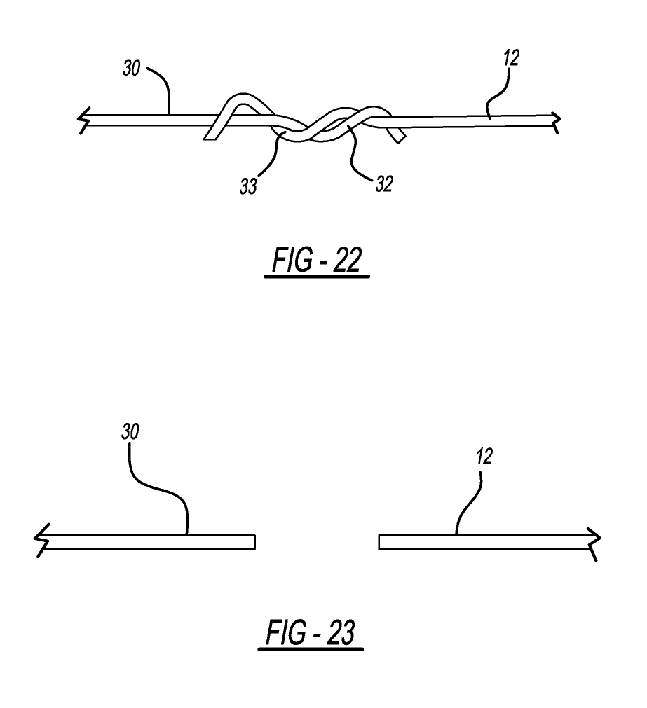

In another approach, shown in FIGS. 22 and 23, the gripper mechanism 30 can include a single arm 32 or wire at its distal end, and the medical device 12 can includes a similar arm or wire 33. One or both of the arm 32 and arm 33 can be made from SMA with high transition temperature and wide hysteresis. The arms 32 and 33 can be coiled together or intertwined, with a remembered shape being straight. Upon reaching transition temperature, one or both of the arms 32 and 33 will straighten, thereby releasing the medical device 12, as shown in FIG. 23.

In the above described, the SMA material has been described as having high transition temperature with a wide hysteresis band. In the case of the gripper mechanism 30 releasing the medical device 12, and where heating of the medical device 12 is not necessary or no longer necessary after it has reached its transition temperature, it is possible for the gripper mechanism 30 to have a more typical and narrow hysteresis band, such that the gripper mechanism 30 will be allowed to close or no longer be subject to its remembered shape after application of heat has ceased. This is possible because the medical device 12 will have already been released and the gripper mechanism 30 may no longer need to stay in its remembered state to release the already released device.

The foregoing description of various embodiments of the invention has been presented for purposes of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1. A medical system for delivering a medical device, the system comprising:
an introducer assembly;
a gripper mechanism coupled to the introducer assembly, the gripper mechanism having both an open state and a closed state;
a resistive element coupled to the introducer assembly and the gripper mechanism, the resistive element configured for being connected to a current source;
wherein the gripper mechanism is at least a portion of the resistive element or is coupled to the resistive element;
a medical device releasably coupled to the gripper mechanism;
wherein the gripper mechanism includes shape memory material, and the gripper mechanism will transition from a martensite state into an austenite state in response to being heated to a final transition temperature via the resistive element, wherein the final transition temperature is higher than a normal human body temperature.

2. The system of claim 1, wherein the medical device is constructed from a shape memory material, the shape memory material having a predetermined deployed shape and being formable into a compressed delivery shape relative to the predetermined deployed shape, wherein the medical device will transition toward the predetermined deployed shape in response to being heated to a final transition temperature and transitioning into an austenite state via the resistive element;
wherein the medical device will remain in the austenite state when heating via the resistive element is ceased.

3. The system of claim 1, wherein the gripper mechanism transitions into the open state in response to being heated via the resistive element, wherein the open state corresponds to the austenite state of the gripper mechanism material.

4. The system of claim 1, wherein the gripper mechanism has a hysteresis band defined between the final transition temperature and a lower temperature at which the material will transition to a martensite state, wherein the final transition temperature is above a normal human body temperature and the lower temperature is below a normal human body temperature.

5. The system of claim 1, wherein the gripper mechanism will not transition into an austenite state when placed within a human body until heating is applied via the resistive element.

6. The system of claim 2, wherein the material of the medical device has a hysteresis band defined between the final transition temperature and a lower temperature at which the material will transition to a martensite state, wherein the final transition temperature is above a normal human body temperature and the lower temperature is below a normal human body temperature.

7. The system of claim 2, wherein the medical device will not transition into an austenite state when placed within a human body until heating is applied via the resistive element.

8. The system of claim 2, wherein the medical device comprises an embolization coil, the embolization coil having a predetermined shape corresponding to a tortuous tangle, wherein the embolization coil transitions to the predetermined shape in response to being heated to the final transition temperature.

9. The system of claim 2, wherein the medical device has a superelastic condition after reaching the final transition temperature, wherein the medical device will bend and flex in response to external forces exerted thereon and will return to the predetermined shape of the austenite state when the external forces are removed.

10. The system of claim 9, wherein the medical device remains in the superelastic condition after heating of the medical device has ceased and the temperature of the device has fallen below the final transition temperature.

11. The system of claim 10, wherein the medical device remains in the superelastic condition at temperatures above a predetermined lower temperature, wherein the predetermined lower temperature is below a normal body temperature.

12. The system of claim 6, wherein the hysteresis band is greater than or equal to 5 degrees C.

13. The system of claim 1, wherein current from a current source passes through the gripper device.

14. The system of claim 1, wherein the gripper mechanism is coupled to a pair of leads, and the gripper mechanism and the leads define the resistive element, and current flows through the leads and through the gripper mechanism, where the resistance of the gripper mechanism is greater than the resistance of the leads, such that the gripper mechanism provides the resistance in the resistance element to heat the gripper mechanism to a temperature higher than the leads.

15. The system of claim 1, wherein the gripper mechanism is a portion of the resistive element, wherein the resistive element further includes a pair of leads, and the leads and the gripper mechanism have the same resistance, such that the gripper mechanism and the leads are heated to approximately the same temperature.

16. A medical device comprising:
an implantable body configured for being attached to an introducer mechanism including a resistive heating element, the implantable body comprising a shape memory material, the body having a predetermined shape in an austenite state and being compressed into a delivery shape in a martensite state;
wherein the shape memory material of the body is constructed to transition to the austenite state at a final transition temperature that is above the normal human body temperature and to remain in the austenite state within a hysteresis band, the hysteresis band defined between the final transition temperature and a lower temperature;
wherein the final transition temperature is above the normal human body temperature and the lower temperature is below the normal human body temperature.

17. The medical device of claim 16, wherein the hysteresis band is 10 degrees C. or greater.

18. The medical device of claim 16, wherein the final transition temperature is above 40 degrees C. and the lower temperature is below 35 degrees C.

19. The medical device of claim 16, wherein the implantable body is heated via current being passed therethrough.

20. A method for implanting a medical device within a body vessel, the method comprising the steps of:
providing an introducer assembly, a gripper mechanism coupled to the introducer assembly, the gripper mechanism having both an open state and a closed state; a resistive element coupled to the introducer assembly, the resistive element configured for being connected to a current source; and a medical device releasably coupled to the gripper mechanism; wherein the gripper mechanism is at least a portion of the resistive element or is coupled to the resistive element and the gripper mechanism includes shape memory material, and the gripper mechanism will transition from a martensite state into an austenite state in response to being heated to a final transition temperature via the resistive element, wherein the final transition temperature is higher than a normal human body temperature;
delivering the gripper mechanism and the medical device to a body vessel;
applying a current to the gripper mechanism and in response thereto, heating the gripper mechanism to the final transition temperature,
in response to heating the gripper mechanism to the final transition temperature, transitioning the gripper mechanism to the open state; and
in response to transitioning the gripper mechanism to the open state, releasing the medical device from the gripper mechanism within the body vessel.

21. The method of claim 20, wherein the medical device is in a compressed delivery state when delivered to the body vessel with the gripper mechanism, wherein the medical device comprises a shape memory material configured to transition from martensite to austenite in response to reaching a final transition temperature, wherein the austenite state corresponds to an expanded delivery state and the final transition temperature is above a normal body temperature;
applying a current to the medical device and in response thereto, heating the medical device to the final transition temperature,
in response to heating the medical device to the final transition temperature, expanding the medical device;
ceasing application of the current to the gripper device and cooling the medical device to the ambient body temperature;
wherein the medical device remains in the austenite state and the expanded deployed state in response to being cooled.

22. The method of claim 21, wherein the medical device will transition back to a martensite state upon being cooled to a lower temperature that is below normal body temperature.

23. The method of claim 22, wherein the difference between the final transition temperature and the lower temperature is greater than 10 degrees C.
